(19) United States
(12) Patent Application Publication
(10) Pub. No.: US 2010/0247448 A1
(43) Pub. Date: Sep. 30, 2010

(54) NOVEL LANTHANIDE LIGANDS AND COMPLEXES, AND USE THEREOF AS CONTRAST AGENTS

(75) Inventors: Christelle Gateau, Champ sur Drac (FR); Marinella Mazzanti, St. Martin Le Vinoux (FR); Aline Nonat, Fontaine (FR)

Correspondence Address: EGBERT LAW OFFICES, 412 MAIN STREET, 7TH FLOOR, HOUSTON, TX, 77002, US

(73) Assignee: COMMISSARIAT A L'ENERGIE ATOMIQUE, Paris (FR)

(21) Appl. No.: 12/161,567

(22) PCT Filed: Jan. 19, 2007
(86) PCT No.: PCT/FR2007/000109

§ 371 Date: Dec. 22, 2008

(30) Foreign Application Priority Data
Jan. 20, 2006 (FR) 0600531

(51) Int. Cl. A61K 49/10; C07D 255/02; C07D 401/14; C07D 403/14; C07F 5/00

(52) U.S. Cl. 424/9.363; 540/474; 540/465

(57) ABSTRACT

The present invention relates to a ligand for metals, in particular lanthanides, of the general formula (I)

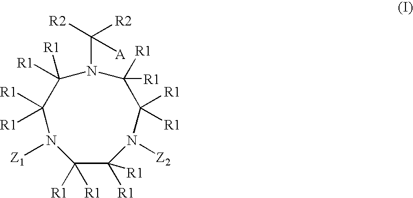

A corresponds to an organic acid radical, to an alkyl or aryl ester. R1 and R2 correspond, individually and independently of one another, to an H, an alkyl radical or an aryl radical. Z1 and Z2, identical or different, are of the general formula (ZA) or (ZB):

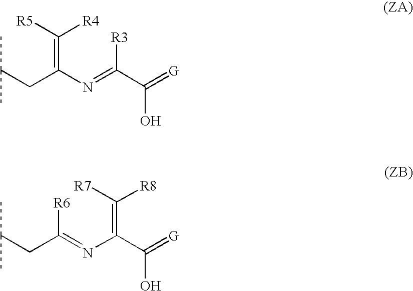

G represents an O, N, P, S or a C substituted independently with an H, an alkyl radical or an aryl radical. R3 to R8 correspond, individually and independently of one another, to an H, an alkyl radical or an aryl radical. The present invention also relates to co-ordinating complexes of the general formula: [Ln(L)(H2O)n] wherein Ln is a lanthanide, L corresponds to a ligand, as well as the grafting thereof to a molecule of interest and their preparation method. The present invention relates moreover to a contrast agent and a pharmaceutical composition including at least one ligand and/or complex and/or molecule of interest.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates in particular to new lanthanide ligands and complexes and their usage as contrast agents, for magnetic resonance and optical imaging in the medical field.

2. Description of Related Art Including Information Disclosed Under 37 CFR 1.97 and 37 CFR 1.98

Magnetic resonance imaging (MRI) is a powerful medical diagnostic technique based on RMN. In order to increase the intensity of the signal and the quality of the images obtained by MRI, contrast agents are used.

The unique spectroscopic and magnetic properties of the lanthanide ions are such that these metals and their complex provide ideal molecules for usage in the medical and biochemical field, and in particular, as contrast agents for magnetic resonance imaging and as optical tracers.

According to the rules set by the <<International Union of Pure and Applied Chemistry>> (IUPAC), by lanthanide is meant the series of the chemical elements ranging from Cerium (Z=58) to lutetium (Z=71).

Whereas the Europium (Eu^{III}) and Terbium (Tb^{II}) complex, with their long life luminescence and their well defined emission spectra, are often used in the design of detectors, as spectroscopic and luminescent probes for solving structural and analytical problems and as fluorescence imager systems, gadolinium (III), with high magnetic torque (S=7/2) and a slow electronic relaxation, is a metal ideal for the design of the relaxing agents for magnetic resonance imaging (MRI).

The design and the structure of a ligand, fundamental for usage in the medical field, are often the subject matter of studies in the field of co-ordination chemistry.

Poly(amino)carboxylate ligands have been studied particularly. Indeed, their high kinetic and thermodynamic stability are essential properties to avoid in vivo toxicity.

Currently, all the GdIII contrast agents commercially available are low molecular weight complexes obtained from poly(amino)carboxylate octadentate ligands, such as, in particular, the 1,4,7,10-tetraazacyclododecane-N,N′,N″,N′″-tetra-acetic (H4dota) acid macrocycle and the diethylene triamine-N,N′,N″,N′″-penta-acetic acid acyclic compound (H5dtpa).

Still, in these complexes, the relaxivity is lower than the possible theoretical maximum, it is due to an absence of simultaneous optimisation simultaneous of all the decisive parameters responsible for the increase in relaxation.

As already mentioned, the key property of the contrast agents is their ‘relaxivity’. Relaxivity is defined as the capacity of a complex to increase the relaxation speed of the protons of the surrounding water molecules. The paramagnetic complexes of gadolinium (III) have set the trend as contrast agents due to the particular electronic and magnetic properties of this ion.

Greater relaxivity is a necessary and essential characteristic for the next generation contrast agents for magnetic resonance imaging (MRI).

Higher relaxivity may be obtained in the presence of a larger number of water molecules, associated with an optimization in the exchange speed of these molecules and with long electronic relaxation and rotational correlation times.

Gadolinium is a very toxic metal in the hydrated form [Gd(H₂O)₉]³⁺. To avoid any in vivo toxicity, it must be used in the form of an inert and thermodynamically stable complex. Moreover, the ligand which complexes the metal must leave co-ordination sites available so that one or several water molecules may link to metal, thereby increasing its relaxivity.

Recently, gadolinium complexes of the ligands exhibiting a “tripod” or “tetrapod” structure with picolinate “arms’ and exhibiting interesting relaxation properties have been published.

However, and although studies have been conducted in order to understand the molecular parameters responsible for relaxivity, the mechanisms and the co-ordination properties underlying the electronic relaxation of the complexes of Gd(III) still remain very poorly understood and known. This prevents and makes the design of new ligands more difficult which exhibit an ideal electronic relaxation then becoming particularly significant for the new generation of macromolecular complexes with long rotational correlation time.

Besides, the preparation of stable and highly in-water light-emitting lanthanide complexes requires the design of polydentate ligands including photosensitizers capable to protect the central metal of the water molecules of the solvent to prevent non-radiative deactivation of the energised states of the lanthanide metal by O—H oscillators.

The N,N′-bis[(6-carboxypyridin-2-yl)methyl]-ethylene diamine-N,N′-diacetic (H4bpeda) acid ligand with a “tetrapod” structure leads to nona-co-ordinated gadolinium complexes which are water soluble and with a water molecule linked with the gadolinium ion. This complex exhibits a water/proton relaxivity and a water exchange rate similar, possibly even a little more favourable, to the commercially available contrast agents and seems to exhibit the quickest electronic transversal relaxation known until now.

Completely different relaxation properties have been observed in the case of the highly symmetrical nona-dentate ligand 1,4,7-tris[(6-carboxypyridin-2-yl)methyl]-1,4,7-triazacyclononane (H₃tpatcn).

The ligand H₃tpatcn leads to highly rigid nona-coordinated gadolinium complex. This complex does not contain any co-ordinated water molecules and exhibits a particularly high low-field-relaxivity. Slow spin electronic relaxation has been estimated from the profile of [Gd(tpatcn)] obtained by nuclear magnetic relaxation (NMR) dispersion complying with detailed studies obtained in paramagnetic electronic resonance (PER) showing, for the case of this complex, the smallest width between bands observed for gadolinium chelates.

The zero-field spin electronic relaxation (approx. 1500 ps) is the greatest value obtained up to this day for this type of complex (650 ps for dota). The slow electronic relaxation of this complex was attributed to the unusual co-ordination sphere including six nitrogenous donor atoms associated with high symmetry.

However, and in spite of the numerous studies performed regarding the preparation of the highly luminescent complexes, the luminescent markers including lanthanides, and currently commercially available, remain seldom due to their difficult preparation.

Moreover, fluorescence imagers and nuclear magnetic resonance imagers, both main non-destructive techniques used in the medical field, exhibit a few shortcomings, in particular low penetration depth into the tissues for the case of fluorescence imagers and a low sensitivity for the case of magnetic resonance imagers.

The bifunctional use of contrast agents for optical imagers and magnetic resonance imagers enables to study the same biological structures at different resolutions and depths.

Due to the different requirements in the design of the bimodal imagers used in both techniques mentioned above, there are today very few examples of molecules used in the synthesis of lanthanide complexes which exhibit simultaneously good magnetic and optical properties.

The aim of the present invention is to provide new lanthanide complexes, which remedy the shortcomings afore-mentioned, particularly as regards their design (bimodal imagers) and their stability in aqueous medium.

Another aim of the present invention is to provide new contrast agents including new structures capable of complexing efficiently the lanthanides efficiently, and particularly gadolinium and terbium.

Another aim of the present invention is to provide new contrast agents including new structures exhibiting a slow electronic relaxation.

Another aim of the present invention is to provide new contrast agents including new structures exhibiting a relaxivity similar to that of the contrast agents commercially available.

Another aim of the present invention is to provide new structures capable, simultaneously of complexing the lanthanides efficiently, with a relaxivity similar to that of the contrast agents commercially available, and having remarkable optical properties.

Another aim of the present invention is to provide new structures having remarkable optical properties.

Other aims and advantages of the invention will appear in the following description, which is given solely for illustrative purposes and without being limited thereto.

### BRIEF SUMMARY OF THE INVENTION

The present invention relates to a ligand for metals, in particular lanthanides, of the general formula (I):

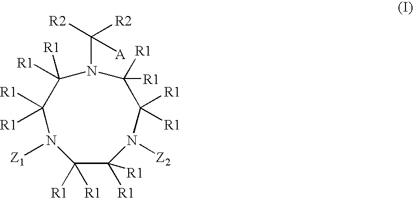

wherein,
A corresponds to an organic acid radical, to an alkyl or aryl ester,
R1 and R2 correspond, individually and independently of one another, to an H, an alkyl radical or an aryl radical,
Z1 and Z2, identical or different, are of the general formula ZA or ZB:

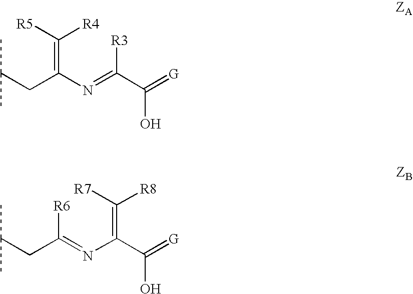

wherein,
G represents an O, N, P, S or a C substituted independently with an H, an alkyl radical or an aryl radical,
R3 to R8 correspond, individually and independently of one another, to an H, an alkyl radical or an aryl radical.

The present invention also relates to a co-ordination complex of the general formula:

[Ln(L)(H₂O)n]

wherein L corresponds to a ligand, according to the present invention, and n is an integer between 0 and 6.

The present invention also relates to a preparation method of ligands and/or complexes and/or molecules of interest as defined above, characterised in that it includes the following steps:
functionalizing two nitrogens of the cycle of a triazacyclononane by Z1 and/or Z2 in the presence of Z1-LG and/or Z₂-LG, in particular by nucleophilic substitution, with LG representing a labile group; and
functionalizing of the triazacyclononane obtained during the previous step by C(R₂)₂A in the presence of LG-C(R₂)₂A, with A representing an alkyl or aryl ester, and R2 corresponding, individually and independently, to an H, an alkyl radical or an aryl radical.

The present invention relates moreover to the ligands and the complexes grafted to a molecule of biological interest, as well the contrast agents and the pharmaceutical compositions including at least one of these molecules.

### BRIEF DESCRIPTION OF THE SEVERAL VIEWS OF THE DRAWINGS

The invention will be understood better when reading the following description, accompanied by the appended drawings.

FIG. 1 represents a schematic view of a diagram of the synthesis of a ligand.

FIG. 2 represents a graph illustration of a standardized titration curve for H₃bpatcn and bpatcn-M.

FIGS. 3a and 3b represent graph illustrations respectively of the emission spectra of [Eu(bpatcn)]⁻ and [Tb(bpatcn)]⁻ after excitation of the ligand at 274 nm.

FIG. 4 represents a graph illustration of the absorption spectrum (......) of H₃bpatcn and the excitation spectrum (--------) of [Tb(bpatcn)] in a buffer solution of Tris.

FIG. 5 represents a schematic view of a diagram of the constitutional balance of the interconversion of the different stereoisomers of [Ln(bpatcn)].

### DETAILED DESCRIPTION OF THE INVENTION

The present invention relates first of all to ligands for metals, in particular lanthanides, of the general formula (I):

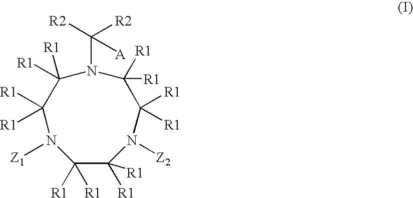

wherein,
A corresponds to an organic acid radical, to an alkyl or aryl ester,
R1 and R2 correspond, individually and independently of one another, to an H, an alkyl radical or an aryl radical,
Z₁ and Z₂, identical or different, are of the general formula (ZA) or (ZB):

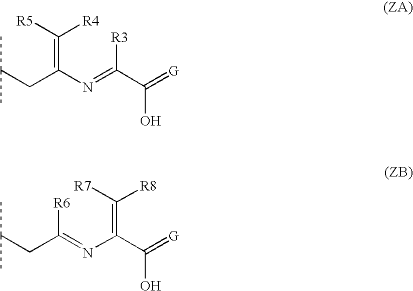

wherein,
G represents an O, N, P, S or a C substituted independently with an H, an alkyl radical or an aryl radical,
R3 to R8 correspond, individually and independently of one another, to an H, an alkyl radical or an aryl radical.

An alkyl radical may be optionally mono- or polysubstituted, linear, branched or cyclic, saturated or unsaturated, bridging or not bridging, in C1-C20, preferably in C1-C6, wherein the substituent(s) may contain one or several heteroatoms such as N, O, F, Cl, P, Si, Br or S. Among such alkyl radicals, the methyl, ethyl, propyl, isopropyl, butyl, isobutyl, tert-butyl and pentyl radicals may be quoted in particular. The ethenyls, propenyls, isopropenyls, butenyls, isobutenyls, tert-butenyls, pentenyls and acetylenyls may also be quoted among the unsaturated alkyl radicals.

An aryl radical may be a mono- or polysubstituted, aromatic or heteroaromatic carbonated structure, formed of one or several aromatic or heteroaromatic cycles each including 3 to 8 atoms, wherein the heteroatom(s) may be N, O, P or S.

Optionally, when the alkyl or aryl radicals are polysubstituted, the substituents may be different from one another. Among the substituents of the alkyl and aryl radicals, The halogen atoms, the alkyl, haloalkyl, aryl, substituted or not, hétéroaryle substituted or not, amino, cyano, azido, hydroxy, mercapto, keto, carboxy, etheroxy and alkoxy such as methoxy groups may be quoted in particular.

In a preferred embodiment A corresponds to a CO₂H, a PO(OH)₂, a PO(OR)(OH), a PO(OR)(OR′), an SO₃H or an SO₂OR with R et R′ representing an alkyl radical or an aryl radical.

Advantageously R₃ and R₄, R₆ and R₇, are two by two bridging alkyl or aryl radicals.

Moreover, and preferably, Z₁ and Z₂ are then selected among the following structures:

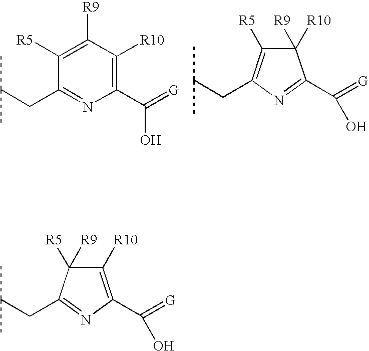

wherein,
R9 and R10 correspond independently to an H, an alkyl radical or an aryl radical.

According to a particular embodiment G represents oxygen. It is particularly advantageous that Z₁ and/or Z₂ comprise at least one moiety liable to confer fluorescence properties to the ligand, wherein such a moiety may be in particular an aromatic moiety, such as an aryl radical.

The present invention also relates to co-ordination complexes of the general formula:

[Ln(L)(H₂O)ₙ]

wherein L corresponds to a ligand as described in the present invention and n is an integer varying from 0 to 6, preferably 1.

In a particular embodiment of the present invention, Ln is a lanthanide, to a three-oxidation degree selected preferably among gadolinium, terbium, europium, neodymium, erbium and ytterbium.

In another particular embodiment of the present invention, the ligands and/or the complexes as described in the present application may moreover be grafted to a molecule of biological interest.

The molecules of interest according to the invention correspond in particular to the biomolecules such as the nucleotides, the polypeptides, the deoxyribonucleic (DNA) and ribonucleic (RNA) acids, the antibodies or any other active molecule of biological and/or medicinal interest. It may be any molecule which an experimenter wishes to detect inside a living system, using in vivo or in vitro techniques, and using the magnetic properties of the metal and the intrinsic optical properties of the metal or the possible fluorescence conferred by the ligand.

Grafting may be provided by any type of link; thus for a lastable grafting it is desirable to make use of a covalent link whereas, for a more reversible grafting, hydrogens interactions may be used.

The present invention also relates to a preparation method of ligands and/or complexes and/or molecules of interest as defined above, characterized in that it includes the following steps:
functionalizing two nitrogens of the cycle of a triazacyclononane by Z1 and/or Z2 in the presence of Z1-LG and/or Z2-LG, in particular by nucleophilic substitution, with LG representing a labile group; and
   functionalizing of the triazacyclononane obtained in (1) by C(R₂)₂A in the presence of LG-C(R₂)₂A, with A representing an alkyl or aryl ester, and R₂ individually and independently as defined above.

In a particular embodiment of the present invention, the labile LG moiety is selected preferably among a halogen, such as Cl or Br, a sulfonate, such as a triflate, a tosylate or a mesylate.

The method may include moreover at least one purifying step, performed in particular by chromatography. The order in which the steps are carried out may be modified, and the functionalization may also start by reaction with LG-C(R₂)₂A.

An additional grafting step to a molecule of interest is advantageous. The molecule of interest may be grafted at numerous points on the ligand or the complex by any suitable reaction from available functions arranged on the complex and in particular from alkyl or aryl radicals present on the structure and as shown above. It is preferable that the grafting is performed with the ligand rather than with the complex.

It is for instance useful to arrange on the structure a function capable of creating a peptidic link for adding a polypeptide. It is thus possible to make use of an acid or alcohol function exhibits on one of the alkyl radicals to enable grafting of dendrons or of moieties liable of interacting with a protein such as albumin, a nucleotic base may be used for grafting on a DNA or an RNA.

In the case of the preparation of a complex, the method includes a step of incorporating of a lanthanide salt performed preferably in aqueous medium in the presence of a lanthanide inorganic salt, such as LnCl₃.6H₂O, and while adjusting the pH to promote the trapping of the lanthanide salt by the complex.

The invention also relates to the contrast agents including at least one complex and/or ligand and/or molecules of interest described above, as well as the contrast agents liable of being obtained from ligands and/or complex and/or molecules of interest defined above.

The invention also corresponds to the use of the ligands and/or co-ordination complexes described above, grafted or not to a molecule of interest, for the preparation of a contrast agent useable in a diagnostic method and in particular a medical imaging method.

Preferably this method makes use of the magnetic and/or fluorescent properties of the complexes employed. The methods affected are in particular magnetic resonance imaging (MRI), solved-time luminescence microscopy, and Fluorescence resonance energy transfer (FRET).

An application to X-ray diffraction imaging and in the development of radiotracers is also contemplated.

The invention also corresponds to the pharmaceutical compositions, including at least one ligand and/or one complex, grafted or not to a molecule of interest and/or a contrast agent as defined previously, useable in a diagnostic method and in particular a medical imaging method.

Moreover the composition may contain any excipient, such as an aqueous solution tolerated by the human and/or animal system, known to the man of the art and useable for conveying the contrast agents.

The invention also relates to the use of at least one ligand and/or one complex and/or one molecule, of interest and/or one contrast agent as defined previously, for the preparation of an pharmaceutical composition useable in a diagnostic method and in particular a medical imaging method.

The diagnostic methods affected by the invention are the imaging methods and more particularly nuclear magnetic resonance imaging and fluorescence imaging.

One of the advantages of the invention lies in that the ligands as defined possess a strong affinity for the lanthanides with respect to the salts, which can be found in biological media such as calcium. This selectivity suggests increased innocuousness of the contrast agents during in vivo usage since it prevents from any transmetallation with salts present in the system such as Ca²⁺.

High luminescence, associated with water solubility with physiological stability, suggest that the complex [Ln(L)H₂Oₙ], such as [Tb(bpatcn)(H₂O)], is adapted for the development of the luminescence imagers for biomedical applications, such as immunological fluorescence tests.

Another advantage of the invention lies in that it is possible to prepare compounds (with different metals) including the same organic molecule which is hence distributed in the same way in the same tissues but which may be detected using different methods. Indeed the presence of at least one aromatic moiety as well as that of a lanthanide salt including a water molecule in the first co-ordination sphere ensure double functionality to these complexes. The same molecule complexed to two different metals (Gd or Tb) will thus enable to study the same biological structure with two different techniques (MRI and luminescence microscopy) and hence with different resolutions and different depths.

Besides, easy functionalization of the ligands, such as bpatcn³⁻, in order to prepare macromolecules as contrast agents with longer correlation times, should enable to study the influence of optimisation of the electronic relaxation on the relaxivity of the systems with high molecular weight.

Moreover, the favourable electronic relaxation properties observed for the gadolinium complex by NMR and PER indicate that, after grafting to a macromolecule, relaxivity greater than that of the commercial contrast agents might be reached.

Finally the straightforward functionalization of the ligands should enable to obtain more hydrophobic compounds for cell imaging and to introduce it in macromolecular systems.

By way of non limiting example, the preparation of the H₃bpatcn ligands and of the Ln(bpatcn) complexes is described above.

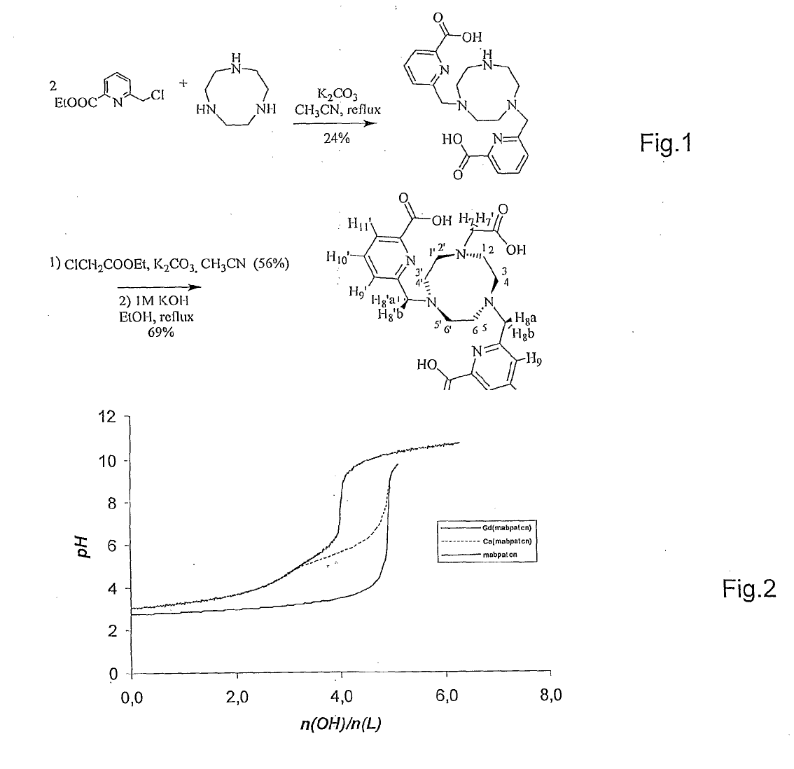

The H₃bpatcn ligand has been prepared, as represented schematically on FIG. 1 as follows.

Under an argon atmosphere, 1,4,7-triazacyclononane trihydrochloride (0.431 g, 1.81 mmol) and K₂CO₃ (1.05 g, 7.62 mmol) are added successively to an ethyl ester solution of 6-chloromethylpyridine-2-carboxylate (0.760 g, 3.81 mmol) in anhydrous acetonitrile (50 mL).

After stirring at room temperature for one hour, the reaction mixture is brought to reflux during 18 hours. The inorganic salts are filtered and the solvent evaporated, then, the product obtained is purified by chromatography on a III-activity alumina column (50 g, CH₂Cl₂/EtOH 100 at 98/2) and 1,4-bis[(6-carbethoxypyridin-2-yl)methyl]-1,4,7-triazacyclononane is obtained in the form of a yellow oil (0.193 g, 24%).

Then, ethyl chloroacetate (0.255 g, 2.08 mmol) and K₂CO₃ (0.288 g, 2.08 mmol) are successively added to a solution of 1,4-bis[(6-carbethoxypyridin-2-yl)methyl]-1,4,7-triazacyclononane (0.860 g, 1.89 mmol) in anhydrous acetonitrile (60 mL).

The reaction mixture is brought to reflux overnight. After filtration and evaporation of the solvent, the product obtained is purified by chromatography on a III-activity alumina column (90 g, CH₂Cl₂/EtOH 100 at 98/2) and the 1-carbethoxymethyl-4,7-bis[(6-carbethoxypyridin-2-yl)methyl]-1,4,7-triazacyclononane is obtained in the form of a yellow oil (0.568 g, 56%).

A solution of KOH 1M (6.5 mL) is added to a solution of 1-carbethoxymethyl-4,7-bis[(6-carbethoxypyridin-2-yl)methyl]-1,4,7-triazacyclononane (0.321 g, 0.570 mmol) in ethanol (10 mL). The reaction mixture is brought to reflux overnight. After evaporation of the solvent, the oil obtained is dissolved in water and the pH is adjusted to 1.5 by addition of an aqueous solution of HCl 1.2M.

After slow evaporation of the solution, the ligand H₃bpatcn.2.5KCl.2HCl.4H₂O is obtained in the form of white crystals (0.310 g, 69%).

The complexes Ln(bpatcn) may be prepared as follows:

A solution of EuCl₃.6H₂O (0.13 mmol) in water (0.4 mL) is added to a solution of H₃bpatcn (0.15 mmol) in water (2 mL). The pH of the solution obtained is adjusted to 7.5 by addition of an aqueous solution of KOH 1M. After evaporation of water, the solid obtained is dissolved in ethanol (20 mL). The suspension obtained is cooled down to 4° C. overnight and KCl is removed by filtration. After slow evaporation of the solution, the complex [Eu(bpatcn)] is obtained in the form of a microcrystalline white solid (66.3 mg, 65%).

The complexes [La(bpatcn)], [Lu(bpatcn)] and [Gd(bpatcn)] are isolated according to the same procedure.

The de-protonation constants of H₃bpatcn defined as Kₐᵢ=[H₆₋ᵢL]³⁻ⁱ/[H₅₋ᵢL]¹⁻ⁱ[H⁺] have been determined by potentiometric titration and exhibit the following values: pKₐ₁=2.2(2), pKₐ₂=2.3(2), pKₐ₃=3.7(3), pKₐ₄=5.42(3) and pKₐ₅=10.5(2) (0.1 M KCl, 298 K).

The titration curves for the case of H₃bpatcn and its Gd^{III} and Ca^{II} complexes are represented on FIG. 2.

The nuclear magnetic resonance (NMR) spectra of the ligands, at different pH's, shows significant variations of the chemical displacement (0.3-0.4 ppm) of both methylenic protons (close to the picolinic and carboxylic acid) during the fifth (pH=10-13) and the fourth (pH=4.5-7) protonations. During the second and the third protonations (pH=1.5-4.5) rather significant variations are observed only for the methylenic protons close to the picolinic moieties. Significant variations are observed in the chemical displacements of the three pyridyl protons (H₃ and, to a lower extent, H₄, H₅, 0.3-0.2 ppm) after the second and the third protonations.

The protonation curves show that both first acid equivalents protonate identically the different types of nitrogen atoms of the macrocycle (pKₐ₄=5.42(3), pKₐ₅=10.5(2)). Both following equivalents protonate the carboxylate moieties associated with the pyridines (pKₐ₂=2.3(2), pKₐ₃=3.71(3).

The value of pKₐ₁ (2.2(2)) matches the value found for the protonation of the carboxylate moiety in the 1,4,7-triazacyclononane-N,N′,NN″-triacetic (H₃nota) acid, ligand (2.88(2)), described by C. F. G. C. Geraldes, M. C. Alpoim, M. P. M. Marques, A. D. Sherry, M. Singh, Inorg. Chem. 1985, 24, 3876-3881.

The crystalline structure of the ligand H₃bpatcn.2HCl protonated isolated at pH˜2 wherein all the carboxylic acids and both nitrogens adjacent to the picolinate moieties of the macrocycle are protonated, also matches the attribution of the pKₐ₁ to the protonation of the carboxylic acid.

The protonation curve and the structural data match a simultaneous partial protonation of the three nitrogens of the macrocycle as observed for the ligand H₃nota, followed by the protonation of the carboxylate moieties. The protonation of the third amine and of the nitrogens of the pyridines takes place at lower pH and the related pKa has not been determined.

Both higher values of pKa are similar to the highest pKa for the case of 1,4,7-triazacyclononane cyclic triamine (10.42 and 6.82) and of the H₃nota triaza macrocycle ligand (11.3(1) and 5.59(2)).

The values of pKₐ₂ and pKₐ₃ match the values found for the protonation of the picolinate moieties in the ligand with “tripod” structure H₃tpaa (H₃tpaa=α, α′, α″ nitrilotri(6-methyl-2-pyridinecarboxylic) acid (pKₐ₂=3.3(1), pKₐ₃=4.11(6)).

Whereas the insertion of the pyridinecarboxylate moieties is responsible for a decrease in the basic character of the ligand H₃tpaa relative to H₃nta (nitrilotriethanoic acid) and of the ligand H₄bpeda relative to H₄edta, the ligand H₃bpatcn exhibits a type of protonation and a pKa value very similar to the parent ligand H₃nota.

The values of pKa and log β for H₄bpdea and for ligands of the same family are shown on the table below

| ligand | pKa | Log βGdL | logβCaL |
|---|---|---|---|
| H₃bpatcn^{a} | 10.5(2); 5.42(3); 3.71(3); 2.3(2); | 15.8(2) | 8.18(7) |
| | 2.2(2) | | |
| H₃nota^{b} | 11.3; 5.6; 2.88 | 13.7 | 8.92 |
| H₄bpdea^{b} | 8.5(1); 5.2(2); 3.5(1); 2.9(1) | 15.1(3) | 9.4(1) |
| H₄edta^{b} | 10.19; 6.13; 2.69; 2.60 | 17.4 | 10.5 |
| H₃nta^{b} | 9.75; 2.64; 1.57 | 11.4 | |
| H₃tpaa^{b} | 4.11(6); 3.3(1); 2.5(2) | 10.2(2) | 8.5(2) |

^{a}present work
^{b}literature

The stability constants of the complexes of H₃bpatcn of Gd^{III} and of Ca^{III} have been calculated by direct titration of a mixture of 1:1 metal:H₃bpatcn (5.10⁻⁴ M) in a pH range between 2.5 and 8.5.

The titration data may be represented by the following equations:

Gd³⁺+bpatcn³⁻⇄[Gd(bpatcn)] log K_{GdL}=15.8(2);

Ca²⁺+bpatcn³⁻⇄[Ca(bpatcn)]⁻ log K_{CaL}=8.18(7)

The values of pGd=13.6 and pCa=6.30 (pM being defined for a metal M as −log [M]_{free} under certain conditions, here pH 7.4, [M]ₜₒₜₐₗ=1 μM and [bpatcn]ₜₒₜₐₗ=10 μM), which enable direct comparison of the stabilities of the complexes under physiological conditions, suggest good stability relative to the commercial contrast agent [Gd(dtpa-bma)(H₂O)]⁻(log K_{GdL}=1.85, pGd=15.8, pCa=6.39).

While the values of pKa and, consequently, the basicity of H₃bpatcn and of H₃nota are very similar, the stability of the gadolinium complex of bpatcn³⁻ is substantially higher than that of the nota complex (log K_{GdL}=13.7).

The additional presence of two electron-donor nitrogen atoms of the pyridyl moiety in bpatch implies an increase in stability (2.1 log units) of the gadolinium complex.

The contribution of the 2-pyridylmethyl to stability has been estimated to 2.6 log units for the case of the complex Gd^{III} with N,N′-bis(2-pyridinylmethyl)ethylenediamine-N,N′-diacetate.

The results described in the present application show that pyridine, contributes substantially to the stability of the gadolinium complex, same when it is part of the 6-methyl-2-pyridinecarboxylic moiety.

Besides, the stability constant of the calcium complex of bpatcn³⁻ is similar to that of nota³⁻. Consequently, the donor moieties N, i.e. the pyridyl moieties in bpatcn³⁻, are responsible for the selectivity of Gd^{III} relative to Ca^{III} in ligands as described above. The high selectivity of the ligand for gadolinium relative to physiological metals is very significant for application of these complexes in magnetic resonance imaging (MRI), since the release of the Gd^{III} associated with in vivo transmetallation is responsible for the toxicity of the gadolinium complex.

The absorption spectra of bpatcn³⁻ and its complexes of Eu^{III} and Tb^{III} show an intense band at ˜36500 cm⁻¹ with a molar absorption coefficient of 9050 for Eu and of 9100 for Tb. These bands have been attributed to a combination of π-->π* and n-->π* centred transitions of the ligand.

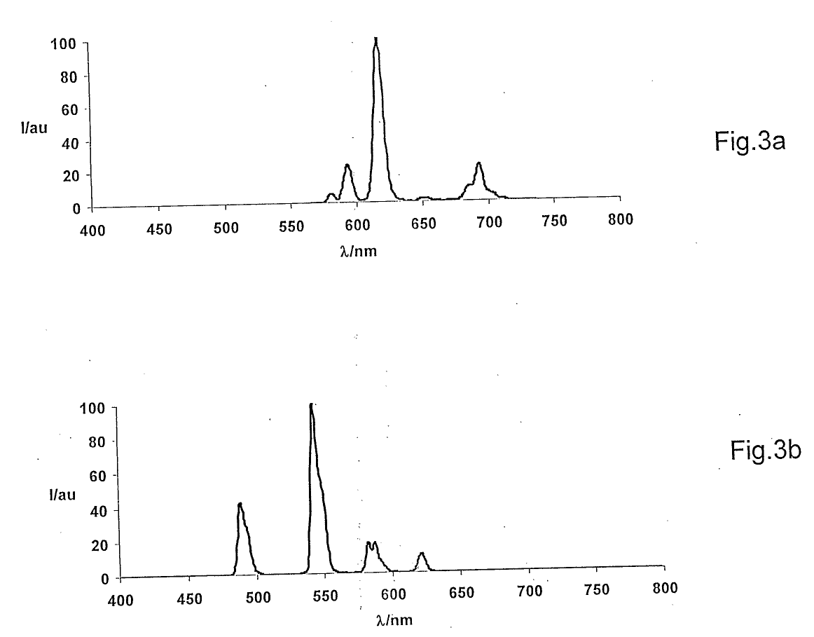

FIGS. 3a and 3b show respectively the emission spectra of the solutions of the europium and terbium complexes at pH=7.4 (obtained after excitation at 273 nm) and the usual and typical transitions ⁵D₀-->⁷F_{J} and ⁵D₄-->⁷F_{J} (J=0-6) of the ions Eu³⁺ and Tb³⁺.

The lanthanide ions Eu and Tb are sensitized efficiently by the ligand bpatcn³⁻ as regards their luminescence properties and, in particular, as regards the emission in the visible zone.

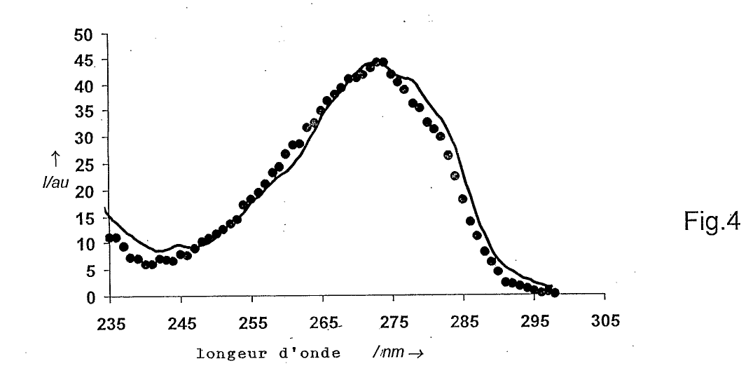

As shown on FIG. 4, an efficient energy transfer between the metal and the ligand is suggested by the similarities between the excitation and absorption spectra of the chelate Tb.

The emission quantal throughput of the complex of [Tb(bpatcn)(H₂O)] (F=43%) calculated relative to [Tb(dpa)₃]³⁻ (H₂dpa=dipicolinic acid) in a buffer solution Tris 0.1M, with 15% experimental error, exhibits one of the highest values found until now and the highest value for the terbium complexes including a water molecule coordinated to the central metal.

The chromophore bpatcn³⁻ sensitizes the ion Eu less efficiently. The complex has less high quantal throughput (F=5%), close to the quantal throughput of commercially available light-emitting probes.

The very intense luminescence of the ion Tb is a consequence of an efficient energy transfer from the ligand towards the metal, and shows that there is an efficient protection of the metal ion relative to a non-radiative deactivation in spite of the presence of a water molecule co-ordinated on the metal.

The long life time of the luminescence observed for the terbium complex in water (1.5 ms), excludes the presence of a de-energizing process including the energy return of the metal in its energized state towards the ligand.

The inefficiency of the non-radiative de-energizing process (most significant in the case of terbium), gives rise to high luminescence quantal throughput for the terbium complex.

The comparison of the luminescence in water and in deuterized water shows that the non-radiative de-energizing process caused by the solvent affects the quantal throughput of the terbium complex far less than the quantal throughput of the europium complex.

The following table shows that the chromophore bpatcn sensitizes the metal Tb very efficiently leading to a quantal throughput value Φ=48%, much higher than the quantal throughput of the terbium complexes existing in the probes commercially available currently and describes in B. Alpha, V. Balzani, J.-M. Lehn, S. Perathoner, and N. Sabbatini, Angew. Chem. Int. Ed. Engl., 1987, 26, 1266; G. Mathis, Clin. Chem., 1993, 39, 195

| | λ_{exc} | | τ_{H2O} | τ_{D2O} | | |
|---|---|---|---|---|---|---|
| compound | (nm) | ε (M⁻¹cm⁻¹) | (ms) | (ms) | Φ_{H2O} | Φ_{D2O} |
| bpatcn | 272 | 7850 | | | | |
| Eu (bpatcn) | 273 | 9050 | 0.542 (4) | 1.67 (4) | 0.05 | 0.12 |
| Tb (bpatcn) | 273 | 9100 | 1.49 (2) | 2.46 (7) | 0.43 | 0.48 |

So as to determine the structure of the lanthanide complexes, the nuclear magnetic resonance (NMR) spectra of the complexes [Ln(bpatcn)] (Ln=La, Eu, Lu) have been studied and compared with results published for the complexes [Ln(dota)]⁻ and lanthanide complexes with ligands including the macrocyclic 1,4,7-triazacyclononane core.

The lanthanide complexes with the ligand bpatcn³⁻ should exhibit 24 signals for the MNR spectrum when all the donor atoms are co-ordinated (symmetry C₁). The rigid co-ordination of the lanthanide ions may give rise to two chirality-independent structural elements associated with the cycle Ln-N—C—C—N and the torsional angles of the “arm” in suspension.

The cycle may exhibit two enantiomeric conformations (λλλ) and (δδδ) and the “arm” may be in the form of a helix either clockwise (Δ) or anticlockwise (Λ).

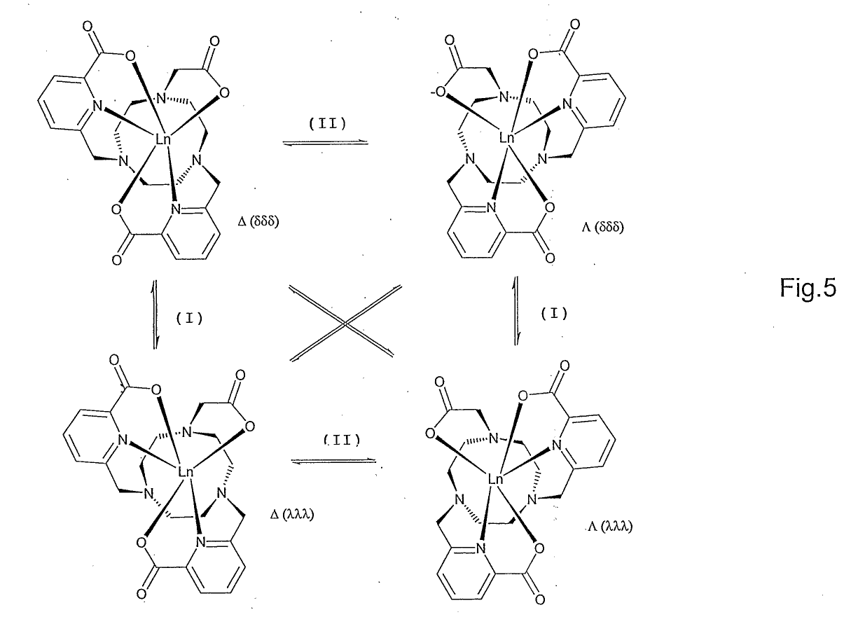

Consequently, two pairs of enantiomers (Δ(λλλ)/Λ(δδδ) or Δ(δδδ)/Λ(λλλ)) of diastereoisomers may be formed and the interconversion by reversal du cycle (I) or by planned rotation of the “arm” (II) may take place, as shown on FIG. 5.

The presence of two pairs of diastereoisomers with a rigid structure sets forth that it gives two groups of 24 signals for the ¹H NMR, while the existence of a rapid exchange process between the enantiomers would give rise to a molecule with a symmetry Cs and exhibiting only 12 signals for the ¹H NMR.

The arrangement of the chelated “arm” in a non-helicoidal shape may also be found in these asymmetric complexes and would also give rise to isomers with symmetry C₁.

Previous studies on lanthanide complexes of the hexadentate trianionic ligand, the triaza 1,4,7-triazacyclononane-N,N′,N″-triacetic (H₄nota) acid in water and those of the 1,4,7-tris(carbamoylmethyl)-1,4,7-triazacyclononane hexadentate neutral ligand in acetonitrile, suggest the presence in these complexes of a triaza flexible core with a rapid interconversion between both conformations of the ring with five members Ln-N—C—C—N.

The proton spectrum MNR of the lanthanum complex in water at a pH=7.1 and at a temperature of 298 K, shows 12 signals with three resonances for the protons of the pyridine, two resonances (doublet) for the moiety CH₂ close to the pyridine, a resonance for the protons of the acetate moiety and six wide resonances superimposed for the protons of the ethylenic half of the macrocycle.

These features match the presence of species exhibiting a symmetry Cs wherein both picolinate “arms” are equivalent.

The diastereotopic character of the CH₂ moiety close to the pyridine can be explained by the fact that the co-ordination of the three nitrogens of the macrocycle exhibits a long lifetime and by the fact that the adjacent quaternary nitrogens exhibit an asymmetric character.

The chemical displacement of the methylene protons of the acetate moiety and of the germinal protons of the ethylenic half of the macrocycle requires conformational mobility of the ligands in solution.

The MNR spectra in water, at a temperature between 298 and 278 K, and in a water-methanol mixture, at a temperature between 278 and 233 K, only show increasingly wide signals preventing any interpretation of the dynamic process taking place.

The ¹H NMR spectrum of the complex bpatcn³⁻ with the metal diamagnetic Lu(III) in a solution of D₂O at pH=4.2 and at a temperature of 298K, only shows a group of 24 signals, with 6 resonances for the protons of the pyridine, 12 resonances (6 axial and 6 equatorial) partially superimposed for the protons of the ethylenic half of the macrocycle, and 6 resonances for the methylenic protons of the “arms”.

An experiment 2D-COSY associated with an experiment ¹H-¹H NOESY has enabled exact attribution of the protons of the pyridine and of the protons of the methylene of the “arms” in suspension. High NOE (Nuclear Overhauser Effect) effect can be observed between the protons H_{8a/8b} and H_{8a′/8b′} of the CH₂ close to the pyridine and the protons H₉ and H_{9′} of the pyridine.

The moieties CH₂—CH₂ of the macrocycle form a complex group of multiplets, doublets of doublets and triplets, which could only be attributed partially.

The diastereotopic character of the methylene protons (quartets AB) of the “arm” and of the protons of the macrocyclic core results from the asymmetric character of the adjacent quaternary nitrogens.

These features match the presence of a rigid structure with a symmetry C₁ in solution wherein the macrocycle and the “arms” remain linked to the NMR time scale. The NMR spectra remain unchanged over a pH range between 4.2 and 9.

In the case of the dota⁴⁻ lanthanide complexes, two isomers can be observed in solution at room temperature and coalescence behavior can be observed for temperatures above 318K, associated with rapid interconversion of both isomers.

Conversely, a single isomer is observed at room temperature for the complex [Lu(bpatcn)] and the ¹H NMR spectrum obtained in D₂O between 278 and 343 K shows a spectrum quasi-unchanged over this temperature range.

Bidimensional EXSY experiments have been conducted in water at 298 K and at 343 K. Whereas no exchange has been detected at room temperature, at 343 K the bidimensional EXSY spectrum in water shows cross-signals indicating an exchange between two species which generates a plane of symmetry.

At 343 K, the ¹H NMR spectrum of the complex of Eu(III) of bpatcn³⁻ in a solution of D₂O and at pH=9.1, shows, as in the case of Lu, a single group of 24 narrow signals with the same intensity matching the presence of the species with a C1-symmetry at that temperature.

The signals have been ascribed completely using a 2D-COSY experiment associated with a 1H-1H NOESY experiment.

The bidimensional EXSY experiment performed at 343 K has shown 12 cross-signals between two groups of protons connected by a plane of symmetry. This can be inferred to the presence in solution of the pairs of enantiomers (Δ(λλλ)/Λ(δδδ) or Δ(δδδ)/Λ(λλλ)) in slow exchange at this temperature.

These results suggest that the metallic ion is situated surrounded by the three “arms” and that the macrocycle remains bound by the five nitrogens and the three oxygens, same at high temperature for the case of europium and of lutetium, as observed previously for the lanthanide complexes of the nona-dentate symmetrical ligand tpatcn. Besides, based upon the NMR studies, a similar arrangement of the donor atoms around the central metal may be anticipated for the lanthanide complexes of tpatcn³⁻ and bpatcn³⁻.

A similar conformation was found in the case of a 1,4,7-triazacyclononane nona-dentate derivative with three iminocarboxylic “arms”.

The same study group (L. Tei, A. J. Blake, M. W. George, J. A. Weinstein, C. Wilson, M. Schröder, Dalton Trans. 2003, 1693-1700) published recently the structure in solid condition and in solution of lanthanide complexes [Ln(L)(CH₃COO)] of a similar derivative, the bis-anionic 1,4,7-triazacyclononane heptadentate with two iminocarboxylic “arms”. While a more flexible structure in solution is observed for these complexes, the co-ordination geometries in solid condition are similar for both ligands.

This is a consequence of the presence of the 1,4,7-triazacyclononane moiety which causes the metallic ion Ln to take on a trigonal prismatic structure.

The proton NMR spectrum of the complex [Eu(bpatcn)(H₂O)] is quite widened when the temperature decreased, suggesting a dynamic process slowing down. Below 283K, the signals become narrower and, at 278 K, the spectrum looks like the spectrum obtained at 343 K, showing the same 24 signals, a little wider and displaced slightly. The NMR spectra have been recorded every 10 K between 343 K and 278 K which enable tracking the evolution of the signals and the attribution of the signals in the spectrum at the lowest temperature.

The nona-co-ordinated gadolinium complex of bpatcn³⁻ shows a relaxivity at imager fields which is similar to that present in the contrast agents commercially available currently [Gd(dota)(H₂O)]⁻ and [Gd(dtpa)(H₂O)]²⁻ and [similar to that observed in the nona-co-ordinated complex of Gd(bpdea)(H₂O)]⁻.

In spite of the presence of co-ordinated water molecules, the molecular architecture of bpatcn³⁻ is responsible for the luminescence of Eu^{III} and Tb^{III} and produces a terbium complex with long lifetime luminescence and with very high quantal throughput.

Its intense luminescence, associated with its water solubility and its water stability, turn the complex [Tb(bpatcn)(H₂O)] into an excellent candidate for the use thereof as a luminescent probe in the biomedical. EPR calculations have enabled to assess the electronic relaxation of this complex with imager fields. The electronic relaxation of the complex of Gd(bpatcn) is slower than that found in the contrast agents commercially available. Such feature should lead to greater relaxivity when the complex is included in a macromolecular system.

The system described above forms one of the existing seldom bifunctional probes and that which exhibits the most intense luminescence.

Naturally, other embodiments, understandable to the man of the art, may be contemplated without departing from the framework of the invention.

1. A ligand for metals, in particular lanthanides, of the general formula (I) 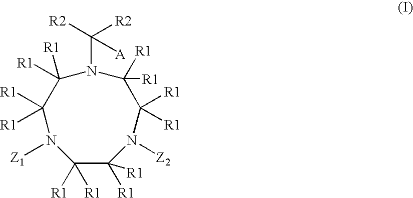
wherein,
A corresponds to an organic acid radical, to an alkyl or aryl ester,
R₁ and R₂ correspond, individually and independently of one another, to an H, an alkyl radical or an aryl radical,
Z₁ and Z₂, identical or different, are of the general formula (Z_{A}) or (Z_{B}): 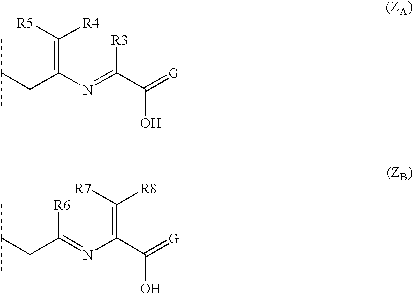
wherein,
G represents an O, N, P, S or a C substituted independently with an H, an alkyl radical or an aryl radical,
R3 to R8 correspond, individually and independently of one another, to an H, an alkyl radical or an aryl radical.

2. A ligand according to claim 1, wherein A corresponds to a CO₂H, a PO(OH)₂, a PO(OR)(OH), a PO(OR)(OR′), an SO₃H or an SO₂OR with R et R′ representing an alkyl radical or an aryl radical.

3. A ligand according to claim 1, wherein R₃ and R₄, R₆ and R₇ are two by two bridging alkyl or aryl radicals.

4. A ligand according to claim 1, wherein Z₁ and Z₂ are selected among the following structures: 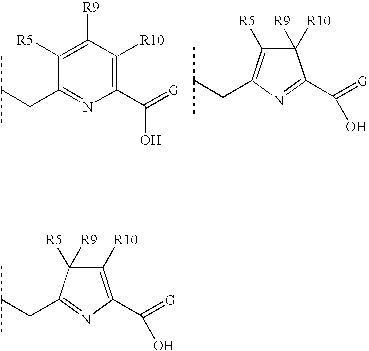
wherein,
R9 and R10 correspond, individually and independently to an H, an alkyl radical or an aryl radical and G represents an O, N, P, S or a C substituted independently with an H, an alkyl radical or an aryl radical.

5. A ligand according to claim 1, wherein G represents an oxygen.

6. A ligand according to claim 1, wherein Z₁ and/or Z₂ comprise at least one moiety liable to confer fluorescence properties to the ligand.

7. A ligand according to claim 6, wherein the moiety is an aromatic moiety.

8. Co-ordinating complexes of the general formula:
[Ln(L)(H2O)ₙ]
wherein Ln is a lanthanide, L corresponds to a ligand according to claim 1 and n is an integer between 0 and 6.

9. Complexes according to claim 8, wherein the lanthanide is selected among gadolinium, terbium, europium, neodymium, erbium and ytterbium.

10. A ligand according to claim 1, being grafted to a molecule of interest.

11. A ligand according to claim 9, wherein the molecule of interest is a biomolecule.

12. A preparation method for ligands and/or complex and/or molecules of interest the method comprising the steps of:
functionalizing two nitrogens of the cycle of a triazacyclononane by Z₁ and/or Z₂ in the presence of Z₁-LG and/or Z₂-LG, with LG representing a labile group, in particular by nucleophilic substitution; and
functionalizing triazacyclononane obtained during the previous step by C(R₂)₂A in the presence of LG-C(R₂)₂A, with A representing an alkyl or aryl ester, and R2 corresponding individually and independently to an H, an alkyl radical or an aryl radical.

13. A contrast agent comprising at least one ligand and/or complex and/or molecule of interest according to claim 1.

14. A pharmaceutical composition comprising at least one ligand and/or a complex, grafted or not to a molecule of interest, and/or a contrast agent according to claim 1.
